(19) United States
(12) Patent Application Publication
(10) Pub. No.: US 2006/0084116 A1
(43) Pub. Date: Apr. 20, 2006

(54) Analysis of MHC-peptide binding interactions

(75) Inventor: Umesh S. Muchhal, Monrovia, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: Xencor, Inc., Monrovia, CA (US)

(21) Appl. No.: 11/226,928

(22) Filed: Sep. 13, 2005

Related U.S. Application Data
(60) Provisional application No. 60/609,885, filed on Sep. 13, 2004.

(51) Int. Cl. G01N 33/53

(52) U.S. Cl. 435/7.1

(57) ABSTRACT

Methods, apparatuses, and compounds for screening or detecting binding of candidate peptides to an MHC construct is provided. A first component including at least one candidate peptides and a second component including at least one MHC construct are contacted. One of the components is immobilized on a solid support. The presence, absence, or quantity of binding of the peptide and said MHC construct is then determined.

This application claims benefit under 35 U.S.C. 119(e) to U.S. Application Ser. Nos. 60/609,885, filed Sep. 13, 2004, which is incorporated herein by reference in its entirety.

### FIELD OF THE INVENTION

Protein arrays (also known as bioarrays) used to study the binding between MHC proteins and peptides are described herein.

### BACKGROUND

Immunogenicity is a complex series of responses to a substance that is perceived as foreign and may include production of neutralizing and non-neutralizing antibodies, formation of immune complexes, complement activation, mast cell activation, inflammation, hypersensitivity responses, and anaphylaxis. Properly modulating the immunogenicity of proteins may greatly improve the safety and efficacy of protein vaccines and protein therapeutics. Furthermore, methods to predict the immunogenicity of novel engineered proteins will be critical for the development and clinical use of designed protein therapeutics. In the case of protein vaccines, the goal is typically to promote, in a large fraction of patients, a robust T cell or B cell-based immune response to a pathogen, cancer, toxin, or the like. For protein therapeutics, however, unwanted immunogenicity can reduce drug efficacy and lead to dangerous side effects. Immunogenicity has been clinically observed for most protein therapeutics, including drugs with entirely human sequence content.

Cellular immunity is mediated by major histocompatibility complex (MHC) proteins. To elicit an immune response, a protein vaccine or therapeutic must productively interact with several classes of immune cells, including antigen presenting cells (APCs), T cells, and B cells. Each of these classes of cells recognize distinct antigen features: APCs express MHC proteins that bind MHC agretopes, or peptides. T cells express T-cell receptors (TCRs) that recognize T-cell epitopes in the context of peptide-MHC constructs, and B cells express MHC molecules and B-cell receptors (BCRs) that recognize B-cell epitopes. Furthermore, uptake by APCs is promoted by binding to any of a number of receptors on the surface of APCs. Finally, particulate protein antigens may be more immunogenic than soluble protein antigens.

Immunogenicity may be dramatically reduced by blocking any of these recognition events. Similarly, immunogenicity may be enhanced by promoting these recognition events. Several factors can contribute to protein immunogenicity, including but not limited to the protein sequence, the route and frequency of administration, and the patient population. Accordingly, modifying these and other factors may serve to modulate protein immunogenicity. Interaction of proteins and their processed peptides with the surface expressed MHC molecules is generally the first determinant in their ability to induce a immune response, and an analysis of this interaction could be used as a predictive and diagnostic tool to assess the “immunogenicity” of a protein.

There is a need to identify peptides that bind MHC proteins, as well as identify MHC polymorphisms that identify specific peptides. Further, there is a need to identify MHC alleles common to specific disease populations, ethnicities, or geographical region. The present application addresses this and other needs.

### SUMMARY

Methods, apparatuses, and compounds for screening or detecting binding of candidate peptides to an MHC construct is provided. A first component including at least one candidate peptides and a second component including at least one MHC construct are contacted. One of the components is immobilized on a solid support. The presence, absence, or quantity of binding of the peptide and said MHC construct is then determined.

In one aspect, the peptides are immobilized on the support to form an array. The array is then exposed to one or more MHC constructs. In one example, the array of peptides is exposed to a library of MHC constructs.

In another aspect, at least one MHC construct is immobilized on the support to form an array. The array is then exposed to one or more peptides. In one example, the array of MHC constructs is exposed to a library of peptides.

In various embodiments, the MHC construct or peptide can be labeled, such as with a fluorophore or fluorescent protein. For example, an MHC construct can include a label and an MHC protein. Similarly, a peptide can include the amino acid sequence of the peptide and a fluorophore.

The MHC construct or peptide can also be exposed to a secondary label, such as an epitope tag.

In another aspect, an MHC construct can include an attachment linker and an MHC protein.

The present methods, apparatuses, and related compositions provided herein have a variety of uses. These uses include specific subsets of MHC proteins that are particularly representative of specific subsets of the human population are also provided. The present methods, apparatuses, and related compositions provided herein also may be used to identify the agretopes in proteins that are responsible for immunogenicity based on MHC binding propensities. The invention also teaches methods for the efficient production of large number of MHC-II constructs required for the aforementioned analysis.

### BRIEF DESCRIPTION OF THE FIGURES

FIG. 1 shows a schematic an embodiment of the MHC proteins of the present invention.

FIG. 2 shows a schematic of several embodiments of MHC constructs of the present invention.

FIG. 3 is a picture of SDS gels of recombinant MHC α-subunit and β-submit expressed in insect cells. HighFive® cells (2×106 in 2 ml) were transfected with 5 ug each of α & β subunit expression constructs (driven by constitutive promoter plE1). Medium harvested after 4 days, and replenished with fresh serum-free medium, again harvested after 2 days (total 6 days post transfection). 5 ul of medium supernatant/lane. Probed with anti-flag and anti-his antibodies).

FIG. 4 is a picture of SDS gels of recombinant MHC α-subunit and β-submit expressed in mammalian cells. 293T™ cells (2×105 in 20 ml) were transfected with 20 ug each of α & β subunit expression constructs (driven by constitutive promoter pCMV). Medium harvested after 4 days, and replenished with fresh medium, again harvested after 2 days (total 6 days post transfection). 5 ul of medium supernatant/lane. Probed with anti-flag and anti-his antibodies).

FIG. 5 shows a schematic of a scale-up of MHC expression and a picture of a SDS gel of a DR4 MHC with two different transfection reagents.

Using the growth adaptability of Hi-5 cells for maximum yield/effort; DR4 MHC with two different transfection reagents, CellFectin (1,3,5) & Insect GeneJuice (2,4,6).

FIG. 6 shows a schematic of the purification of recombinant MHC constructs. The MHC proteins may be purified using a modular purification protocol that yields>50% pure and concentrated preparation. This is stable and directly usable in binding assays. This coomassie blue stained SDS-PAGE gel shows the purified MHCs of DR class showing the two bands representing α and β subunits for each.

FIG. 7 shows a picture of an SDS gel of expression of multiple DRs using the modular constructs in insect cells. The yields are very comparable to DR4 example for ˜80% of the DRs tested. The supernatants from cells transfected with listed DR constructs were analyzed by western blotting using a mixture of anti-his and anti-flag antibodies (two bands representing the tagged α and β subunits).

FIG. 8 shows a diagraph of a MHC-peptide binding assay.

FIG. 9 shows a graph of a peptide-binding assay showing the specific and competitive binding of biotinylated HA peptide to recombinant DR4 and DR1. Various concentrations (25 to 400 nM) of two different batches of DR4 and DR1 were incubated with 400 nM of biotin-HA peptide with or without 10 fold molar excess of unlabelled HA peptide (C) in a 50 ul reaction volume. The MHC bound bHA peptide was quantitated using Eu-streptavidin time resolved fluorescence assay.

FIG. 10 is a schematic of a method for testing a therapeutic protein on a MHC bioarray of the present invention.

FIG. 11 shows a schematic of a method for testing an array of peptides.

### DETAILED DESCRIPTION

Methods, apparatuses, and compounds for screening or detecting binding of candidate peptides to an MHC construct is provided. A first component including at least one candidate peptides and a second component including at least one MHC construct are contacted. One of the components is immobilized on a solid support. The presence, absence, or quantity of binding of the peptide and said MHC construct is then determined.

In one embodiment, the methods provide for the rapid and facile creation of MHC protein bioarrays that may be used in a wide variety of methods and techniques. The MHC proteins can be immobilized on the surface. These MHC bioarrays may then be used in a wide variety of ways, including diagnosis (e.g. detecting the presence of specific peptides or agretopes), and screening (e.g. looking for target analytes that bind to specific proteins or detecting immunogenicity).

In another embodiment, the present methods allow the rapid and facile creation of peptide bioarrays that may be used in a wide variety of methods and techniques. By immobilizing peptides on the array, the MHC construct targets that bind the peptide may be “captured” on the bioarray.

In another embodiment, the present methods allow for competition between a peptide in an MHC molecule and a second, free peptide. Either the bound peptide or free peptide can be labeled.

In a certain embodiments, the set of MHC proteins assembled in array format is particularly representative of a population of subjects who may be treated with the therapeutic protein of interest. For example, a set of MHC proteins would be those that are found most frequently within the general population (or as a good proxy, those found within the general US population) can be used. In other situations, the intended patient population for a particular therapeutic protein may possess certain MHC alleles more frequently than others. Such populations can include a specific disease population (e.g. it is well established that the class II MCH allele DRB1*1501 is frequently possessed by patients with multiple sclerosis) or a particularly ethnicity that is predisposed to a disease for genetic or geographical reasons. Selection of frequently represented target population alleles for array format will greatly expedite the experimental analysis of the protein, increasing feasibility, data quality, and reducing time and cost. Once a target population of subjects is identified, MHC allele frequencies can either be determined directly by genotyping the patients, or by using existing data regarding the prevalence of MHC alleles within that population. The MHC alleles with the highest frequencies would then be produced and displayed in a readable array. Peptides representative of the protein sequence would then be analyzed for interaction with the arrayed MHC proteins in order to determine the presence of potential MHC agretopes within the protein. In a preferred embodiment, MHC alleles that have higher than 5% frequency within a target population will be arrayed. In alternative embodiments, the array size itself will determine the number of alleles—i.e. if the array holds 96 elements, the 96 highest frequency alleles from the target population could be arrayed.

In additional embodiments of the invention, the choice of arrayed MHC proteins would be influenced by the knowledge that a peptide from the protein does indeed interact with one or more of the MHC proteins. That MHC protein and related MHC proteins expected to have similar peptide binding preferences would then be assembled in array format for evaluating the offending peptide and variants thereof (e.g. variants designed to remove the ability to interact with MHC molecules).

In some embodiments, MHC proteins selected for arrayed format will be a combination of high frequency alleles in a specific target population and high frequency alleles in the general population or a combination of high frequency alleles in a specific target population and alleles expected to interact with peptides within the therapeutic protein.

A. MHC Constructs

1. MHC Proteins

MHC proteins generally come in two separate classes designated class I and class II. The molecules are generally designated by antigenic subtype. Human MHC class I molecules, also referred to as human leukocyte antigens (HLA), are designated HLA-A, -B, and -C. Human MHC class II molecules are designated HLA-DR, -DQ, and -DP.

MHC class I molecules are found on almost every nucleated cell of the body. MHC class I molecules are heterodimers that have a single transmembrane polypeptide chain (the α-chain) and a β₂ microglobulin. The a chain has two polymorphic domains, α₁, α₂, which binds peptides derived from cytosolic proteins. Because MHC class I molecules present peptides derived from cytosolic proteins, the pathway of MHC class I presentation is often called the cytosolic or endogenous pathway.

MHC class I molecules are loaded with peptides generated in the cytosol. As viruses infect a cell by entering its cytoplasm, this cytosolic, MHC class I-dependent pathway of antigen presentation is the primary way for a virus-infected cell to signal T cells. MHC class I molecules generally interact exclusively with CD8⁺ (“cytotoxic”) T cells (CTLs). The fate of the virus-infected cell is almost always apoptosis initiated by the CTL, effectively reducing the risk of infecting neighboring cells.

MHC Class II molecules are found only on a few specialized cell types, particularly antigen-presenting cells (APCs) such as macrophages, B cells, and T cells. Like MHC class I molecules, class II molecules are also heterodimers, but in this case consist of two homologous peptides, an α and β chain. The peptides presented by class II molecules are derived from extracellular proteins. MHC class II molecules bind peptides in a groove between the α and β chains. Because the peptide-binding groove of MHC class II molecules is open at both ends, the peptides presented by MHC class II molecules are generally between 15-24 amino acid residues long. Class II molecules interact exclusively with CD4⁺ (“helper”) T cells (T_{H}S). The helper T cells then help to trigger an appropriate immune response.

As used herein, “MHC construct” means the portion of an MHC class I or class II lacking the transmembrane portion of membrane bound MHC class I and class II proteins. MHC constructs are further capable of functioning as a capture binding ligand immobilized on a solid surface. Likewise, the MHC constructs may function as a target molecule when in solution. MHC class I constructs include a binding pocket that is closed on both ends. The MHC class I constructs are capable of binding a peptide 8-9 amino acids in length. Similarly, MHC class II constructs include a binding pocket that is open on both ends, and capable of binding peptides between, for example, 14 and 25 amino acids long.

B. Peptides and Agretopes

MHC class I and II molecules both bind peptides in their respective binding pockets. A peptide derived from a processed antigen is referred to as an “agretope.” Peptides corresponding to agretopes can be screened according to the methods disclosed herein

Screening methods for the elucidation of binding of candidate peptides and MHC constructs. Candidate peptides include a peptide being tested for activity, e.g. binding to an MHC construct. By “peptide” herein is meant at least two covalently attached amino acids. Generally, MHC class I peptides are 8 or 9 amino acids in length, but can vary to between 7 and 10 amino acids in length. MHC class II peptides can vary from 15 to 24 amino acids in length. Optionally, they can vary from 10 amino acids to 30 amino acids or more in length.

The peptide may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations. Peptide inhibitors of enzymes find particular use.

In one embodiment, the candidate peptides are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

Alternatively, the candidate peptides can comprise randomized peptides, either fully randomized or they are biased in their randomization. By “randomized” or grammatical equivalents herein is meant that each peptide consists of at least a portion of essentially random amino acids, respectively. In some embodiments, the library is biased. That is, some positions within the sequence are either held constant, or are selected from a limited number of possibilities. For example, in one embodiment, the amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

The peptide length can be biased towards peptides that interact with known classes of molecules, such as MHC proteins. Thus, for example, libraries can be generated that have homology to known MHC binding peptides.

By “library” herein is meant a plurality of molecules. In the case of peptides, in some embodiments, the library provides a sufficiently structurally diverse population of peptides to effect a probabilistically sufficient range of cellular responses to provide one or more cells exhibiting a desired response. Accordingly, an interaction library must be large enough so that at least one of its members will have a structure that gives it affinity for some molecule, protein, or other factor whose activity is necessary for completion of the signaling pathway. Although it is difficult to gauge the required absolute size of an interaction library, nature provides a hint with the immune response: a diversity of 107-108 different antibodies provides at least one combination with sufficient affinity to interact with most potential antigens faced by an organism. Published in vitro selection techniques have also shown that a library size of 107 to 108 is sufficient to find structures with affinity for the target. A library of all combinations of a peptide 7 to 20 amino acids in length, such as proposed here for expression in retroviruses, has the potential to code for 207 (109) to 2020 Thus, in a preferred embodiment, at least 106, preferably at least 107, more preferably at least 108 and most preferably at least 109 peptides are simultaneously analyzed in the subject methods. Libraries can be designed to maximize library size and diversity.

As above, the peptides may be linked to a fusion partner, alternatively with primary labels.

The peptides can also be selected based on agretopes. Methods of identifying, adding or remove class I or class II MHC agretopes have been described. For example, vaccines may be made that are more effective at inducing an immune response by inserting agretopes with increased affinity for MHC class I or class II molecules (see for example, WO 9833523; Sarobe, P., et al. J. Clin. Invest., 102:1239-1248 (1998); Thimme, R., et al. J. Virology, 75:3984-3987 (2001); Roberts, C., et al., Aids Research and Human Retroviruses, 12: 593-610 (1996); Kobayashi, H., et al., Cancer Res., 60: 5228-5236 (2000); Keogh, E., et al., J. Immunology, 167: 787-796 (2001); Want, R-F., Trends in Immunology, 22: 269-276 (2001); Mucha et al. BMC Immunol. 3: 1-12 (2002), all incorporated entirely by reference). Removal of MHC agretopes for the purpose of decreasing protein immunogenicity has also been disclosed (for example WO 98/52976, WO 02/079232, WO 00/34317, and WO 02/069232, all incorporated entirely by reference). Addition or removal of MHC agretopes is a tractable approach for immunogenicity modulation because the factors affecting binding are reasonably well defined, the diversity of binding sites is limited, and MHC molecules and their binding specificities are static throughout an individual's lifetime. As immunogenicity may significantly affect the safety and efficacy of protein therapeutics and protein vaccines, methods to evaluate the immunogenicity of designed proteins intended for use as drugs or vaccines would be useful.

Identification of class I MHC-Binding Agetopes

Peptides can be used as either the capture binging ligand or target molecule. Class I MHC constructs, for example, primarily bind fragments of intracellular proteins that are derived from infecting viruses, intracellular parasites, or internal proteins of the cell; proteins that are overexpressed in cancer cells are of special interest. The resulting peptide-MHC constructs are transported to the surface of the APC, where they may interact with T cells via TCRs. This is the first step in the activation of a cellular program that may lead to cytolysis of the APC, secretion of lymphokines by the T cell, or signaling to natural killer cells. The interaction with the TCR is dependent on both the peptide and the MHC molecule. MHC class I molecules show preferential restriction to CD8+ cells, (for example, Fundamental Immunology, 4th edition, W. E. Paul, ed., Lippincott-Raven Publishers, 1999, Chapter 8, pp 263-285), incorporated entirely by reference.

The factors that determine the affinity of peptide-class I MHC interactions have been characterized using biochemical and structural methods, including sequencing of peptides and natural peptide libraries extracted from MHC proteins. Class I MHC ligands are generally octa- or nonapeptides (also known as 8-mers or 9-mers); they bind a groove in the class I MHC structure framed by two α-helices and a β-pleated sheet. Specific pockets in the binding groove recognize subsets of residues in the peptide, called anchor residues; these interactions confer some sequence selectivity. Class I MHC molecules also interact with atoms in the peptide backbone. The orientation of the peptides is determined by conserved side chains of the MHC I protein that interact with the N- and C-terminal residues in the peptide.

Any of a number of methods may be used to identify potential class I MHC agretopes, including but not limited to the computational and experimental methods described below. Rules for identifying MHC I binding sites have been described in Altuvia, Y., et al (1997) Human Immunology, 58:1-11; Meister, GE, et al (1995) Vaccine: 6:581-591; Parker, K. C., et al., (1994) J. Immunology, 152:163; Gulukota, K., et al., (1997) J. Mol. Biol., 267:1258-1267; Buus, S., (1999) Current Opinion Immunology, 11:209-213; all incorporated entirely by reference). Databases of MHC binding peptide, such as SYPEITHI and MHCPEP may also be used to identify potential MHC I binding sites (Rammensee, H-G., et al., (1999) Immunogenetics, 50:213-219; Brusic, V., et al., (1998) Nucleic Acids Research, 26:368-371), all incorporated entirely by reference. Other methods for identifying MHC binding motifs include allele-specific polynomial algorithms described by Fikes, J., et al., WO 01/41788, neural net (Gulukota, K, supra), polynomial (Gulukota, K., supra) and rank ordering algorithms (Parker, K. C., supra), all incorporated entirely by reference.

Identification of class II MHC-Binding Agretopes

Class II MHC molecules, which are related to class I MHC molecules, primarily present extracellular antigens. Relatively stable peptide-MHC constructs may be recognized by TCRs; this recognition event is required for the initiation of most antibody-based (humoral) immune responses. MHC class II molecules show preferential restriction to CD4+ cells (Fundamental Immunology, 4th edition, W. E. Paul, ed., Lippincott-Raven Publishers, 1999, Chapter 8, pp 263-285, incorporated entirely by reference).

The factors that determine the affinity of peptide-class II MHC interactions have been characterized using biochemical and structural methods. Peptides bind in an extended conformation bind along a groove in the class II MHC molecule. While peptides that bind class II MHC molecules are typically approximately 12-25 residues long, a nine-residue region is responsible for most of the binding affinity and specificity. The peptide-binding groove may be subdivided into “pockets”, commonly named P1 through P9, where each pocket comprises the set of MHC residues that interacts with a specific residue in the peptide. Between two and four of these positions typically act as anchor residues. As in the class I ligands, the non-anchoring amino acids play a secondary, but still significant role (Rammensee, H., et al., (1999) Immunogenetics, 50:213-219, incorporated entirely by reference). A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used, including but not limited to, those described in a recent review (Schirle et al. J. Immunol. Meth. 257: 1-16 (2001), incorporated entirely by reference) and those described below.

In one embodiment, structure-based methods are used. For example, methods may be used in which a given peptide is computationally placed in the peptide-binding groove of a given MHC molecule and the interaction energy is determined (for example, see WO 98/59244 and WO 02/069232). Such methods may be referred to as “threading” methods.

Alternatively, purely experimental methods may be used. Examples of physical methods include high affinity binding assays (Hammer, J., et al. (1993) Proc. Natl. Acad. Sci. USA, 91:4456-4460; Sarobe, P. et al. (1998) J. Clin. Invest., 102:1239-1248), T cell proliferation and CTL assays (WO 02/77187, Hemmer, B., et al., (1998) J. Immunol., 160:3631-3636); stabilization assays, competitive inhibition assays to purified MHC molecules or cells bearing MHC, or elution followed by sequencing (Brusic, V., et al., (1998) Nucleic Acids Res., 26:368-371), all incorporated entirely by reference.

In a preferred embodiment, potential MHC II binding sites are identified by matching a database of published motifs, such as SYFPEITHI (Rammensee, H., et al., (1999) Immunogenetics, 50:213-219; or MHCPEP (Brusic, B., et al., supra), both incorporated entirely by reference. Sequence-based rules for identifying MHC II binding sites, including but not limited to matrix method calculations, have been described in Sturniolo, T, et al. Nat. Biotechnol., 17:555-561 (1999); Hammer, J. et al., Behring. Inst. Mitt., 94: 124-132 (1994); Hammer, J. et al., J. Exp. Med., 180:2353-2358 (1994); Mallios, R. R J. Com. Biol., 5:703-711. (1998); Brusic, V., et al., Bioinformatics, 14:121-130 (1998); Mallios, R. R. Bioinformatics, 15:432-439 (1999); Marshall, K. W., et al., J. Immunology, 154:5927-5933 (1995); Novak, E. J., et al., J. Immunology, 166:6665-6670 (2001); Cochlovius, B., et al., J. Immunology, 165:4731-4741 (2000); and by Fikes, J., et al., WO 01/41788), all incorporated entirely by reference.

In an especially preferred embodiment, the matrix method is used to calculate MHC-binding propensity scores for each peptide of interest binding to each allele of interest. The matrix comprises binding scores for specific amino acids interacting with the peptide binding pockets in different human class II MHC molecule. It is possible to consider all of the residues in each 9-mer window; it is also possible to consider scores for only a subset of these residues, or to consider also the identities of the peptide residues before and after the 9-residue frame of interest. The scores in the matrix may be obtained from experimental peptide binding studies, and, optionally, matrix scores may be extrapolated from experimentally characterized alleles to additional alleles with identical or similar residues lining that pocket. Matrices that are produced by extrapolation are referred to as “virtual matrices”. (See Sturniolo, T., Bono, E., Ding, J., Raddrizzani, L., Tuereci, O., Sahin, U., Braxenthaler, M., Gallazzi, F., Protti, M. P., Sinigaglia, F., and Hammer, J. (1999) “Generation of tissue-specific and promiscuous HLA ligand databases using DNA micro arrays and virtual HLA class 11 matrices” Nat. Biotech., 17, 555-61 (1999), all incorporated entirely by reference.)

The virtual matrix approach allows one to predict the MHC-peptide binding propensities, however the predictions are based on several assumptions. It would be best to be able to experimentally characterize each peptide-MHC interaction for any given protein and the population of large number of MHC molecules. In practice, however this becomes a very huge experimental challenge. The present invention teaches methods for efficient production of a large number of MHC molecules and MHC constructs, and the analysis of peptide-MHC interactions using high-throughput array based tools.

C. Fusion Partners

In one embodiment, one or both of the components of the assay (e.g. the MHC construct or the peptide) is linked to a fusion partner. By “fusion partner” herein is meant a sequence that is associated with the component, that confers a common function or ability. Fusion partners can be heterologous (i.e. not native to the host cell), or synthetic (not native to any cell). Suitable fusion partners include, but are not limited to: a) presentation structures, as defined below, which provide the peptides in a conformationally restricted or stable form b) targeting sequences, defined below, which allow the localization of the component into a subcellular or extracellular compartment; c) rescue sequences as defined below, which allow the purification or isolation of either component; d) stability sequences, which confer stability or protection from degradation to the peptide, for example resistance to proteolytic degradation; e) dimerization sequences, to allow for peptide dimerization; or f) any combination of a), b), c), d), and e), as well as linker sequences as needed.

In a some embodiments, the fusion partner is a presentation structure. By “presentation structure” or grammatical equivalents herein is meant a sequence, which, when fused to assay components, causes the attached proteins and peptides to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures.

While the assay components may be either MHC constructs or peptides, presentation structures are preferably used with the MHC constructs or peptides. Thus, synthetic presentation structures, i.e. artificial polypeptides, are capable of presenting a randomized peptide as a conformationally-restricted domain. Generally such presentation structures comprise a first portion joined to the N-terminal end of the randomized peptide, and a second portion joined to the C-terminal end of the peptide; that is, the peptide is inserted into the presentation structure, although variations may be made, as outlined below. To increase the functional isolation of the peptide, the presentation structures are selected or designed to have minimal biologically activity when expressed in the target cell or synthesized de novo.

Some presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In a some embodiments, the presentation structure is a coiled-coil structure, allowing the presentation of the randomized peptide on an exterior loop. See, for example, Myszka et al., Biochem. 33:2362-2373 (1994), hereby incorporated by reference. In a some embodiment, the presentation structure is a minibody structure. A “minibody” is essentially composed of a minimal antibody complementarity region. The minibody presentation structure generally provides two randomizing regions that in the folded protein are presented along a single face of the tertiary structure. See for example Bianchi et al., J. Mol. Biol. 236(2):649-59 (1994), and references cited therein, all of which are incorporated by reference). In a some embodiments, the presentation structure is a sequence that contains generally two cysteine residues, such that a disulfide bond may be formed, resulting in a conformationally constrained sequence.

In a some embodiments, the fusion partner is a rescue sequence (similar to a “secondary label” as described herein). Thus, for example, peptide rescue sequences include purification sequences such as the His6 tag for use with Ni affinity columns and epitope tags. Suitable epitope tags include myc (for use with the commercially available 9E10 antibody), the BSP biotinylation target sequence of the bacterial enzyme BirA, flu tags, lacZ, and GST.

In some embodiments, the fusion partner is a stability sequence to confer stability to the assay component or the nucleic acid encoding it. Thus, for example, peptides may be stabilized by the incorporation of glycines after the initiation methionine (MG or MGGO), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate peptide structure. Thus, some stability sequences are as follows: MG(X)nGGPP (SEQ ID NO: 1), where X is any amino acid and n is an integer of at least four.

In one embodiment, the fusion partner is a dimerization sequence. A dimerization sequence allows the non-covalent association of one random peptide to another random peptide, with sufficient affinity to remain associated under normal physiological conditions. This effectively allows small libraries of random peptides (for example, 104) to become large libraries if two peptides per cell are generated which then dimerize, to form an effective library of 108 (104×104). It also allows the formation of longer random peptides, if needed, or more structurally complex random peptide molecules. The dimers may be homo- or heterodimers.

Dimerization sequences may be a single sequence that self-aggregates, or two sequences, each of which is generated in a different retroviral construct. That is, nucleic acids encoding both a first random peptide with dimerization sequence 1, and a second random peptide with dimerization sequence 2, such that upon introduction into a cell and expression of the nucleic acid, dimerization sequence 1 associates with dimerization sequence 2 to form a new random peptide structure.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods.

The fusion partners may be placed anywhere (i.e. N-terminal, C-terminal, internal) in the structure as the biology and activity permits.

In a some embodiments, the fusion partner includes a linker or tethering sequence. Linker sequences between the fusion partner and the other components of the constructs (such as the randomized MHC constructs or peptides) may be desirable to allow the MHC constructs or peptides to interact with their target unhindered. For example, when the assay component is a peptide, useful linkers include glycine-serine polymers (including, for example, (GS)n, (GSGGS)n (SEQ ID NO: 2) and (GGGS)n (SEQ ID NO: 3), where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are some since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

In addition, the fusion partners, including presentation structures, may be modified, randomized, and/or matured to alter the presentation orientation of the randomized expression product. For example, determinants at the base of the loop may be modified to slightly modify the internal loop peptide tertiary structure, which maintaining the randomized amino acid sequence.

In general, labels may be either direct or indirect detection labels, sometimes referred to herein as “primary” and “secondary” labels. By “detection label” or “detectable label” herein is meant a moiety that allows detection. Accordingly, detection labels may be primary labels (i.e. directly detectable) or secondary labels (indirectly detectable; this is analogous to a “sandwich” type assay). In general, labels fall into four classes: a) isotopic labels, which may be radioactive or heavy isotopes; b) magnetic, electrical, thermal labels; c) colored or luminescent dyes or moieties; and d) binding partners. Labels can also include enzymes (horseradish peroxidase, etc.) and magnetic particles.

In a preferred embodiment, the detection label is a primary label. A primary label is one that may be directly detected, such as a fluorophore. Preferred labels include chromophores or phosphors but are preferably fluorescent dyes or moieties. Fluorophores may be either “small molecule” fluores, or proteinaceous fluores. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”: see U.S. Ser. No. 09/315,584, hereby incorporated by reference), pyrene, Malacite green, stilbene, Lucifer Yellow, Cascade Blue®, Texas Red, Cy dyes (Cy3, Cy5, etc.), alexa dyes, phycoerythin, bodipy, and others described in the 6th Edition of the Molecular Probes Handbook by Richard P. Haugland, incorporated entirely by reference.

In this embodiment, the test molecule is labeled with a primary label. As will be appreciated by those in the art, this may be done in a wide variety of ways, depending on the test molecule. In some cases, primary labels are added chemically using functional groups on the label and the test molecule. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups may be attached to secondary labels comprising amino groups, for example using linkers as are known in the art; for example, homo-or hetero-bifunctional linkers as are well known (see 1994 Pierce Chemical Company catalog, technical section on cross linkers, pages 155-200, incorporated by reference).

In some systems, for example when the test molecule is a protein, the test molecule may be fused to a label protein such as GFP, using well-known molecular biology techniques. Similarly, when the test molecule is a nucleic acid, fluorophores or other primary or secondary labels may be added to any number of the nucleotides using well-known techniques.

In a preferred embodiment, a secondary label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; nuclease inhibitors, enzymes such as horseradish peroxidase, alkaline phosphatases, lucifierases, cell surface markers, etc.

In a preferred embodiment, the secondary label is a binding partner pair. For example, the label may be a hapten or antigen, which will bind its binding partner. For example, suitable binding partner pairs include, but are not limited to: antigens and antibodies (including fragments thereof (FAbs, etc.)); proteins and small molecules (including biotin/streptavidin); enzymes and substrates or inhibitors; other protein-protein interacting pairs; receptor-ligands; and carbohydrates and their binding partners. Nucleic acid—nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx reagents. In one embodiment, the binding partner may be attached to a solid support to allow separation of components containing the label and those that do not.

In a preferred embodiment, the binding partner pair comprises a primary detection label (for example, attached to the test molecule) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁴-10⁶ M⁻¹, with less than about 10⁵-10⁹ M⁻¹, being preferred and less than about 10⁷-10⁹ M⁻¹ being particularly preferred.

In a preferred embodiment, the secondary label is a chemically modifiable moiety. In this embodiment, labels comprising reactive functional groups are incorporated into the test molecule. The functional group can then be subsequently labeled (e.g. either before or after the assay) with a primary label. Suitable functional groups include, but are not limited to, amino groups, carboxy groups, maleimide groups, oxo groups and thiol groups, with amino groups and thiol groups being particularly preferred. For example, primary labels containing amino groups may be attached to secondary labels comprising amino groups, for example using linkers as are known in the art; for example, homo-or hetero-bifunctional linkers as are well known (see 1994 Pierce Chemical Company catalog, technical section on cross linkers, pages 155-200, incorporated by reference).

In some embodiments, the techniques outlined herein result in the addition of a detectable label to the test molecule, which binds to at least one of the candidate proteins (e.g., MHC constructs on a bioarray). Fluorescent labels are preferred, and standard fluorescent detection techniques can then be used.

In other embodiments, detection can proceed with unlabeled test molecules when a “solution binding ligands” or “soluble binding ligands” or “signaling ligands” or “signal carriers” or “label probes” or “label binding ligands” are used. In these embodiments, the soluble binding ligand carries the label and will bind to the test molecule. For example, when proteinaceous test molecules are used, they may be fused to heterologous epitope tags, which can then bind labeled antibodies to effect detection. A wide variety of epitope tags are known as outlined above.

In some embodiments, MHC constructs are added to bioarrays comprising arrays of capture probes, under conditions that allow the formation of binding complexes between the capture sequences of the MHC constructs to the capture probes of the bioarray. This forms the protein arrays of the invention.

The term “label” means any detectable label. Examples of suitable labels include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the label is coupled to the antigen binding protein via spacer arms of various lengths to reduce potential steric hindrance. Various methods for labelling proteins are known in the art and may be used in performing the present invention.

The covalent attachment of the fluorescent label may be either direct or via a linker. In one embodiment, the linker is a relatively short coupling moiety, that is used to attach the molecules. A coupling moiety may be synthesized directly onto a MHC construct or peptide for example, and contains at least one functional group to facilitate attachment of the fluorescent label. Alternatively, the coupling moiety may have at least two functional groups, which are used to attach a functionalized MHC construct or peptide to a functionalized fluorescent label, for example. In an additional embodiment, the linker is a polymer. In this embodiment, covalent attachment is accomplished either directly, or through the use of coupling moieties from the agent or label to the polymer. In a preferred embodiment, the covalent attachment is direct, that is, no linker is used. In this embodiment, the MHC construct or peptide preferably contains a functional group such as a carboxylic acid which is used for direct attachment to the functionalized fluorescent label. Thus, for example, for direct linkage to a carboxylic acid group of a MHC construct or peptide, amino modified or hydrazine modified fluorescent labels will be used for coupling via carbodiimide chemistry, for example using 1-ethyl-3-(3-dimethylaminopropyl)-carbodiimide (EDC) as is known in the art (see Set 9 and Set 11 of the Molecular Probes Catalog, supra; see also the Pierce 1994 Catalog and Handbook, pages T-155 to T-200, both of which are hereby incorporated by reference). In one embodiment, the carbodiimide is first attached to the fluorescent label, such as is commercially available.

Thus, in a preferred embodiment, a fluorescent label is attached, either directly or via a linker, to the MHC constructs or peptides and thus serves as a first labeling moiety. Alternatively, in a preferred embodiment, the first labeling moiety comprises a first partner of a binding pair, which may or may not be fluorescent, and a second labeling moiety, comprising the second partner of a binding pair, and at least one fluorescent label, as defined above.

Alternatively, a secondary label may be used. The secondary label includes a primary label covalently attached to a molecule capable of binding to the MHC construct—peptide complex. Examples include MHC specific antibodies.

Attachment of MHC Constructs or Peptides to Solid Supports

In one embodiment, the bioarrays comprise a substrate. By “substrate” or “solid support” or other grammatical equivalents herein is meant any material appropriate for the attachment of capture probes and is amenable to at least one detection method. As will be appreciated by those in the art, the number of possible substrates is very large. Possible solid supports include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica based materials including silicon and modified silicon, carbon, metals, inorganic glasses, plastics, ceramics, and a variety of other polymers. In a some embodiments, the solid supports allow optical detection and do not themselves appreciably fluoresce. In addition, as is known the art, the solid support may be coated with any number of materials, including polymers, such as dextrans, acrylamides, gelatins, agarose, etc. Exemplary solid supports include silicon, glass, polystyrene and other plastics and acrylics.

Generally the solid support is flat (planar), although as will be appreciated by those in the art, other configurations of solid supports may be used as well, including the placement of the probes on the inside surface of a tube, for flow-through sample analysis to minimize sample volume.

The size of the array can depend on the composition and end use of the array. Arrays containing from about 2 different capture probes to many thousands may be made. Generally, the array will comprise from two to as many as 100,000 or more, depending on the size of the pads, as well as the end use of the array. Preferred ranges are from about 2 to about 10,000, with from about 5 to about 1000 being preferred, and from about 10 to about 100 being particularly preferred. In some embodiments, the compositions of the invention may not be in array format; that is, for some embodiments, compositions comprising a single capture probe may be made as well. In addition, in some arrays, multiple substrates may be used, either of different or identical compositions. Thus for example, large arrays may comprise a plurality of smaller substrates.

In one embodiment, the bioarray substrates optionally comprise an array of capture probes. By “capture probes” herein is meant proteins (e.g. antibodies) or chemicals (attached either directly or indirectly to the substrate as is more fully outlined below) that are used to bind the MHC constructs or peptides. As will be appreciated by those in the art, the capture probes may be attached either directly to the substrate, or indirectly, through the use of polymers or through the use of microspheres.

Once generated, the library of solid supports containing a library of covalently attached MHC constructs or peptides is added to at least a first population of a first target molecule. By “target molecule” herein is meant a molecule for which an interaction is sought; this term will be generally understood by those in the art. Suitable target molecules include, but are not limited to, proteins such as receptors, enzymes, cell-surface receptors, G-protein coupled receptors, ion channels, transport proteins, transcription factors, vesicle proteins, adhesion proteins, etc.

In some embodiments, components of the invention are linked together with attachment linkers. For example, an MHC construct or peptide can be attached to the solid support using an attachment linker, or an MHC protein can be attached to a label with an attachment linker, etc. In general, attachment will generally be done as is known in the art, and will depend on the composition of the two materials to be attached. In general, attachment linkers are utilized through the use of functional groups on each component that can then be used for attachment. Preferred functional groups for attachment are amino groups, carboxy groups, oxo groups, hydroxyl groups and thiol groups. These functional groups can then be attached, either directly or indirectly through the use of a linker. Linkers are well known in the art; for example, homo-or hetero-bifunctional linkers as are well known (see 1994 Pierce Chemical Company catalog, technical section on cross-linkers, pages 155-200, incorporated herein by reference). Preferred attachment linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred, with the corresponding functionalities.

In a preferred embodiment, the attachment linkers facilitate covalent attachment. By “covalently attached” herein is meant that two moieties are attached by at least one bond, including sigma bonds, pi bonds and coordination bonds. In some cases, for example when thiol groups are used to attach components to a gold surface, the thiol-gold attachment is considered covalent under these conditions.

Alternatively, non covalent attachment can be done, for example through the absorption of MHC constructs to the solid supports of the invention.

As is outlined herein, it is also possible to attach proteins using recombinant methods. For example, as is more fully outlined herein, the use of fluorescent proteins as the label for MHC constructs can be done by ligating the encoding nucleic acids together for expression of fusion proteins.

In a preferred embodiment, the MHC constructs or peptides are synthesized first, and then covalently attached to the solid supports. As will be appreciated by those in the art, this will be done depending on the composition of the MHC constructs or peptides and the solid supports. The functionalization of solid support surfaces such as certain polymers with chemically reactive groups such as thiols, amines, carboxyls, etc. is generally known in the art. Generally, the MHC constructs or peptides are attached using functional groups on the MHC construct or peptide. For example, MHC constructs or peptides containing carbohydrates may be attached to an amino-functionalized support; the aldehyde of the carbohydrate is made using standard techniques, and then the aldehyde is reacted with an amino group on the surface. In an alternative embodiment, a sulfhydryl linker may be used. There are a number of sulfhydryl reactive linkers known in the art such as SPDP, maleimides, α-haloacetyls, and pyridyl disulfides (see for example the 1994 Pierce Chemical Company catalog, technical section on cross-linkers, pages 155-200, incorporated herein by reference) which can be used to attach cysteine containing proteinaceous agents to the support. Alternatively, an amino group on the MHC construct or peptide may be used for attachment to an amino group on the surface. For example, a large number of stable bifunctional groups are well known in the art, including homobifunctional and heterobifunctional linkers (see Pierce Catalog and Handbook, pages 155-200). In an additional embodiment, carboxy groups (either from the surface or from the MHC construct or peptide) may be derivatized using well known linkers (see the Pierce catalog). For example, carbodiimides activate carboxy groups for attack by good nucleophiles such as amines (see Torchilin et al., Critical Rev. Therapeutic Drug Carrier Systems. 7(4):275-308 (1991), expressly incorporated herein). Similarly, a number of homo- and heterobifunctional agents are known for amine-amine crosslinking, thiol-thiol crosslinking, amine-thiol crosslinking, amine-carboxylic acid crosslinking, and carbohydrate crosslinking to amines and thiols; see Molecular Probes Catalog, 1996, Sixth Edition, chapter 5, hereby incorporated by reference. In addition, proteinaceous MHC constructs or peptides may also be attached using other techniques known in the art, for example for the attachment of antibodies to polymers; see Slinkin et al., Bioconj. Chem. 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994), all of which are hereby expressly incorporated by reference). It should be understood that the MHC constructs or peptides may be attached in a variety of ways, including those listed above. What is important is that manner of attachment does not significantly alter the functionality of the MHC construct or peptide; that is, the MHC construct or peptide should be attached in such a flexible manner as to allow its interaction with its corresponding peptide or MHC construct.

In general, it is desirable to have a library of MHC constructs or peptides attached to solid supports. By “library of MHC constructs or peptides” herein is meant generally at least about 10² different compounds, with at least about 10³ different compounds being preferred, and at least about 10⁴, 10⁵ or 10⁶ different compounds being particularly preferred.

In general, it is preferred that each solid support contain a multiplicity of MHC constructs or peptides. That is, each solid support will contain at least about 10 MHC constructs or peptides, with at least about 100 being preferred, and at least about 1000 being especially preferred.

As will be appreciated by those in the art, each solid support may contain one type of MHC construct or peptide, or more than one. That is, in a preferred embodiment, any single solid support contains a single type of candidate peptide. This may be preferred for a variety of reasons, including synthetic considerations, ease of characterization of downstream “hits”, and fluorescent detection limits.

Alternatively, (for example when libraries of naturally occuring compounds are attached to solid supports), each solid support may contain more than one type of MHC construct or peptide. In this embodiment, as is more fully outlined herein, it will generally be desirable to “amplify” the fluorescent signal (i.e. have more than one fluorescent label per target) to facilitate detection.

In a preferred embodiment, there are a number of solid supports that each contain a single MHC construct or peptide. That is, there are a number of solid supports each containing a particular MHC construct or peptide. Thus, at least about 100 solid supports per MHC construct or peptide are used, with at least about 1000 being preferred and at least about 10,000 to 100,000 being especially preferred.

Thus, the library of candidate peptides are contained upon a plurality of solid supports.

Arrays

The terms “array” and “bioarray” herein are synonymous, and mean a plurality of capture binding ligands on a solid support. The size of the array will depend on the composition and end use of the array. As discussed above, first example of a bioarray is an array of MHC constructs. A second example of a bioarray is an array of peptides. The biomolecules in the array may be attached to a solid support, free in solution, deposited on a solid support, etc.

In a preferred embodiment, the non-immobilized MHC construct includes at least a first fluorescent label. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Suitable optical dyes are described in the 1996 Molecular Probes Handbook by Richard P. Haugland, hereby expressly incorporated by reference.

In a preferred embodiment, all the labeled MHC constructs or peptides contain the same fluorescent label. In an alternative embodiment, the labeled MHC construct or peptide population is divided into at least two subpopulations, each comprising a different fluorescent label. This may be particularly preferred to reduce false positives; that is, only solid supports comprising both labels (i.e. solid supports with a single MHC construct or peptide type that bind targets with both labels) will constitute “real” interactions.

In one embodiment, the target molecules are also bound to solid supports. In a preferred embodiment, the target molecules are attached to the solid supports using preferably flexible linkers, to allow for interaction with solid support-bound agents. In this embodiment, a preferred system utilizes fluorescent solid supports; that is, the solid support to which the target molecules is attached can be fluorescent, thus serving as the first or second labeling moiety. See for example the Molecular Probes catalog, supra, chapter 6, hereby incorporated by reference.

The solid supports containing the MHC constructs or peptides are added to the target molecules under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away.

Array Formats

The arrays can have a number of formats in which either the the MHC construct or peptides are immobilized on a solid surface to form an array.

In one format, a single MHC construct is immobilized at multiple locations on a solid surface to form an array. The single MHC construct can correspond, for example, to a single MHC allele. The MHC allele is immobilized to the solid surface as described supra. The array can then be exposed to one or more peptides. For example, the peptides can be spotted onto the surface at each location in the array. Alternatively, a pool of different peptides can be applied to the array of MHC constructs. The peptides can be added to the array, and those that bind can be detected.

Alternatively, a plurality of MHC constructs are immobilized at various locations on the solid surface to form an array. The MHC constructs can, for example, correspond to a plurality of known alleles for a specific type of MHC. Alternatively, the MHC constructs can correspond to multiple different MHC subtypes of MHC molecules, such as a combination of class I and class II molecules, or subtypes thereof. The array can then be exposed to one or more peptides, such as a pool of peptides.

In another format, an array of peptides may be provided. For example, a single peptide can be provided at multiple locations on the solid surface to form an array. The single peptide can correspond to a specific peptide, including a specific agretope presented at the surface of specific MHC molecules. The peptide can be designed to bind in the binding groove of a subset of MHC alleles in a specific class or antigen subtype (e.g. HLA-A, B, C, DR, DQ, DP).

In the peptide array format, the peptide is immobilized to the solid surface as described supra. The array can then be exposed to one or more MHC construct in solution form. For example, a single MHC allele can be provided. Alternatively, the MHC molecules can by within a specific HLA antigen subtype (e.g. HLA-A, B, C, DR, DQ, or DP) can be provided. Alternatively, a pool of different peptides can be applied to the array of MHC constructs. The peptides can be added to the array, and those that bind can be detected.

A variety of other reagents may be included in the assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Once a binding event has been detected, the MHC construct may be identified. Since the location and sequence of each capture probe is known, the identification of a “hit” at a particular location will identify the particular MHC construct with the corresponding capture sequence. This capture sequence may be used to identify the coding region of the candidate protein. This may be done in a wide variety of ways, as will be appreciated by those in the art, including using PCR technologies. For example, using primers specific to the capture sequence, the nucleic acid encoding the candidate protein may be amplified and sequenced.

In a preferred embodiment, the process may be used reiteratively. That is, the sequence of a candidate protein is used to generate more candidate proteins. For example, the sequence of the protein may be the basis of a second round of (biased) randomization, to develop agents with increased or altered activities. Alternatively, the second round of randomization may change the affinity of the agent. Furthermore, if the candidate protein is a random peptide, it may be desirable to put the identified random region of the agent into other presentation structures, or to alter the sequence of the constant region of the presentation structure, to alter the conformation/shape of the candidate protein.

The methods of using the present inventive library can involve many rounds of screenings in order to identify a nucleic acid of interest. For example, once a nucleic acid molecule is identified, the method may be repeated using a different target. Multiple libraries may be screened in parallel or sequentially and/or in combination to ensure accurate results. In addition, the method may be repeated to map pathways or metabolic processes by including an identified candidate protein as a target in subsequent rounds of screening.

In a preferred embodiment, the methods and compositions of the invention comprise a robotic system. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which may be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In some preferred embodiments, the instrumentation will include a detector, which may be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluroescence resonance energy transfer (FRET), SPR systems, luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems, for cell culture growth and transformation in multi-well plates or tubes and for hazardous operations. The living cells will be grown under controlled growth conditions, with controls for temperature, humidity, and gas for time series of the live cell assays. Automated transformation of cells and automated colony pickers will facilitate rapid screening of desired cells.

Flow cytometry or capillary electrophoresis formats may be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which may be done using any number of known heating and cooling systems, including Peltier systems.

In a preferred embodiment, the robotic apparatus includes a central processing unit that communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

The above-described methods of screening bioarrays are based on the determining the immunogenicity of the candidate protein. The sequence or structure of the candidate proteins does not need to be known. A significant advantage of the present invention is that no prior information about the candidate protein is needed during the screening, so long as the product of the identified coding nucleic acid sequence has biological activity, such as specific association with a targeted chemical or structural moiety. The identified nucleic acid molecule then may be used for understanding cellular processes as a result of the candidate protein's interaction with the target and, possibly, any subsequent therapeutic or toxic activity.

The methods described above and their modifications may be used for analyzing the immunogenicity of various proteins in a rapid manner and may be used as a diagnostic tool. Alternatively, the invention may also be used as a tool to create a database of all binding interactions possible for developing better predictive algorithms.

Generally, in a preferred embodiment of the methods herein, one of the components of the invention is non-diffusably bound to an insoluble support having isolated sample receiving areas (e.g. a microtiter plate, an array, etc.). The component bound may be an envelope virus particle expressing the candidate protein or the target molecule, etc. The insoluble support may be made of any composition to which the assay component may be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon®, etc. Microtiter plates and arrays are especially convenient because a large number of assays may be carried out simultaneously, using small amounts of reagents and samples. Alternatively, bead-based assays may be used, particularly with use with fluorescence activated cell sorting (FACS). The particular manner of binding the assay component is not crucial so long as it is compatible with the reagents and overall methods of the invention, maintains the activity of the composition and is non-diffusible. One preferred method of binding include the use of antibodies, more preferably antibodies which do not sterically block either the ligand binding site or activation sequence when the protein is bound to the support. Other preferred methods includes direct binding to “sticky” or ionic supports, chemical crosslinking, the use of labeled components (e.g. the assay component is biotinylated and the surface comprises strepavidin, etc.), the synthesis of the target on the surface, etc. Following binding of the candidate protein or target molecule, excess unbound material is removed by washing. The sample receiving areas may then be blocked through incubation with bovine serum albumin (BSA), casein or other innocuous protein or other moiety.

In a preferred embodiment, the target molecule is bound to the support, and an envelope virus particle expressing a candidate protein is added to the assay. Alternatively, the envelope virus particle expressing a candidate protein is bound to the support and the target molecule is added. Novel binding agents include specific antibodies, non-natural binding agents identified in screens of chemical libraries, peptide analogs, etc. Of particular interest are screening assays for agents that have a low toxicity for human cells. Determination of the binding of the target and the candidate protein may be done using a wide variety of assays, including, but not limited to labeled in vitro protein-protein binding assays, electrophoretic mobility shift assays, immunoassays for protein binding, the detection of labels, functional assays (phosphorylation assays, etc.) and the like.

The determination of the binding of the candidate protein to the target molecule may be done in a number of ways. In a preferred embodiment, one of the components, preferably the soluble one, is labeled, and binding determined directly by detection of the label. For example, this may be done by attaching the envelope virus particle expressing a candidate protein to a solid support, adding a labeled target molecule (for example a target molecule comprising a fluorescent label), washing off excess reagent, and determining whether the label is present on the solid support. This system may also be run in reverse, with the target (or a library of targets) being bound to the support and envelope viruses expressing candidate proteins, preferably comprising a primary or secondary label, added. For example, envelope virus particles expressing a candidate protein comprising fusions with GFP or a variant may be particularly useful. Various blocking and washing steps may be utilized as is known in the art. As will be appreciated by those in the art, it is also possible to contact the envelope viruses expressing the candidate proteins and the targets prior to immobilization on a support.

One embodiment includes a bioarray for nucleic acid binding proteins. The nucleic acid targets may be on the array and envelope virus particles expressing candidate proteins may be added. Similarly, protein bioarrays of libraries of target proteins may be used, with labeled envelope virus particles expressing candidate proteins added. Alternatively, the libraries of virus particles may be attached to the bioarray, either through the nucleic acid or through the protein components of the system. See also U.S. application Ser. No. 09/792,630, filed Feb. 22, 2001, entirely incorporated by reference.

In another embodiment, the bioarray may also be done using bead based systems. For example, for the detection of nucleic acid binding proteins, standard “split and mix” techniques, or any standard oligonucleotide synthesis schemes, assays may be run using beads or other solid supports such that libraries of sequences are made. The addition of envelope virus libraries then allows for the detection of candidate proteins that bind to specific sequences.

In a preferred embodiment, the binding of the candidate protein is determined through the use of competitive binding assays. In this embodiment, the competitor is a binding moiety known to bind to the target molecule such as an antibody, peptide, binding partner, ligand, etc. Under certain circumstances, there may be competitive binding as between the target and the binding moiety, with the binding moiety displacing the target.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound. Similarly, ELISA techniques are generally preferred. In some embodiments, only one of the components is labeled. In an alternate embodiment, more than one component may be labeled with different labels.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, co-factors such as cAMP, ATP, etc., may be used. The mixture of components may be added in any order that provides for the requisite binding.

Screening for agents that modulate the activity of the target molecule may also be done. As will be appreciated by those in the art, the actual screen will depend on the identity of the target molecule. In a preferred embodiment, methods for screening for a candidate protein capable of modulating the activity of the target molecule comprise the steps of adding an envelope virus particle expressing a candidate protein to a sample of the target, as above, and determining an alteration in the biological activity of the target. “Modulation” or “alteration” in this context includes an increase in activity, a decrease in activity, or a change in the type or kind of activity present. Thus, in this embodiment, the candidate protein should both bind to the target (although this may not be necessary), and alter its biological or biochemical activity as defined herein. The methods include both in vitro screening methods, as are generally outlined above, and ex vivo screening of cells for alterations in the presence, distribution, activity or amount of the target.

In a preferred embodiment, bioarrays of the present invention may be designed for specific populations of individuals. Populations may be based upon race, geographic area, sex, disease, etc. Examples of populations also include individuals with the following indications: arthritis, psoriatic arthritis, ankylosing spondylitis, spondyloarthritis, spondyloarthropathies, rheumatoid arthritis, juvenile rheumatoid arthritis, juvenile idiopathic arthritis, reactive arthritis (Reiter Syndrome) scleroderma, Sjogren's syndrome, keratoconjunctivitis, keratoconjunctivitis sicca, TNF-receptor associated periodic syndrome (TRAPS), periodic fever, periprosthetic osteolysis, apthous stomatitis, pyoderma gangrenosum, uveitis, reticulohistiocytosis, inflammatory bowel diseases, sepsis and septic shock, Crohn's Disease, psoriasis, autoimmune thyroiditis, dermatitis, atopic dermatitis, eczematous dermatitis) graft versus host disease (GVHD), hematologic malignancies, such as multiple myeloma (MM), refractory MM, Waldenstrom's macroglobulinemia, myelodysplastic syndrome (MDS) acute myelogenous leukemia (AML); solid tumor malignancies, such as ovarian carcinoma, melanoma, renal cell carcinoma; and the inflammation associated with tumors, pain, including spinal disk pain, chronic lower back pain chronic neck pain, pain due to bone metastasis, pain and swelling after molar extraction, neurological conditions and neural damage conditions such as peripheral nerve injury, demyelinating diseases, adrenoleukodystrophy, X-linked adrenoleukodystrophy (X-ALD), the childhood cerebral form (CCER) and the adult form, adrenomyeloneuropathy (AMN), adrenoleukodystrophy, sciatica, autoimmune sensorineural hearing loss, chronic inflammatory demyelinating polyneuropathy (CIDP), Alzheimers disease, Parkinson's disease, diabetes, insulin resistance, insulin sensitivity, Syndrome X, Wegener's Granulomatosis, dermatomyositis, histicytosis, polymyositis, cancer cachexia, temporomandibular disorders, refractory ocular sarcoidosis, sarcoidosis, behcet's, churg-strauss syndrome, asthma, idiopatic pneumonia following bone marrow transplantation, systemic lupus erythematosus (SLE), lupus nephritis, multiple sclerosis (MS), amyotrophic lateral sclerosis (ALS) myasthenia gravis, atherosclerosis, polyneuropathy, orangomegaly, endocrinopathy, M protein, skin changes (POEMS syndrome), Sneddon-Wilkinson disease, necrotizing crescentic glomerulonephritis, renal amyloidosis, AA amyloidosis, erythema nodosum leprosum (ENL), chronic kidney disease, malnutrition, inflammation and atherosclerosis (MIA) syndrome, chronic obstructive pulmonary disease (COPD), pulmonary fibrosis, endometriosis, idiopathic thrombocytopenic purpura (ITP), AIDS, HIV disease and related conditions, including tuberculosis (TB) in AIDS patients, inflammation and cancer (e.g. Kaposi's Sarcoma, HIV retinopathy, uveitis, P jiroveci pneumonia (PCP), Pneumocystis choroiditis, HIV-associated lymphoma), alopecia greata, allergic responses due to arthropod bite reactions, aphthous ulcer, iritis, conjunctivitis, keratoconjunctivitis, ulcerative colitis, asthma, allergic asthma, cutaneous lupus erythematosus, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens Johnson syndrome, idiopathic sprue, lichen planus, Graves ophthalmopathy, sarcoidosis, primary biliary cirrhosis, and interstitial lung fibrosis.

### EXAMPLES

### Example 1

### Production of MHC-II Molecules

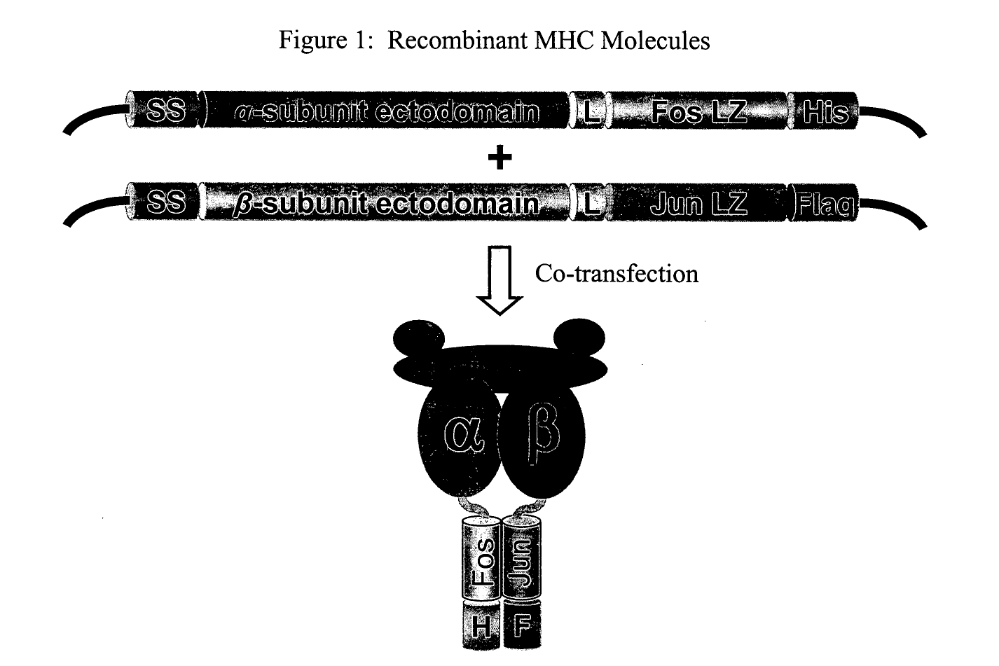

The extracellular peptide binding domains are expressed with C-terminal leucine zippers to facilitate dimerization. See FIGS. 1 & 2. The constructs may include C-terminal purification tags, for example, 6xhis, flag, c-myc, etc. Additional sequences may be attached or modifications made to provide anchoring to a solid surface may be used. These sequences or modifications preferably added at the C-terminus. Examples of such sequences and modifications include but are not limited to biotinylation of the constructs, fusions with the construct (e.g., Fc, albumin, etc.), linker sequences of from 3 to 50 amino acids (preferably combinations of Gly and Ser) or modification with small molecules, all to enhance anchoring the construct to a solid surface.

To facilitate the production of a large number of MHCs the expression constructs encoding both α and β-subunits are co-transfected in production cell lines in transient manner. The preferred expression cells include HighFive, Sf9 or Drosophila S2 insect cells, or for mammalian expression, the 293T cells are preferred. Stable cell lines may also be established for production of selected MHCs. For purification, the affinity chromatography using either the specific tag or anti-MHC antibodies are preferred methods.

### Example 2

### Expression in Insect Cells

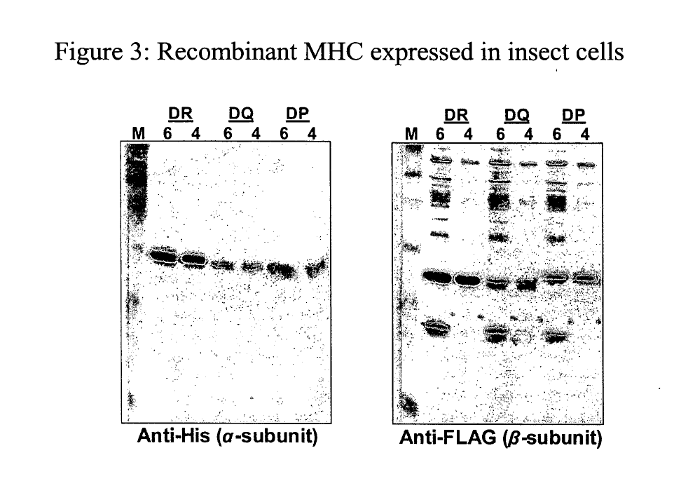

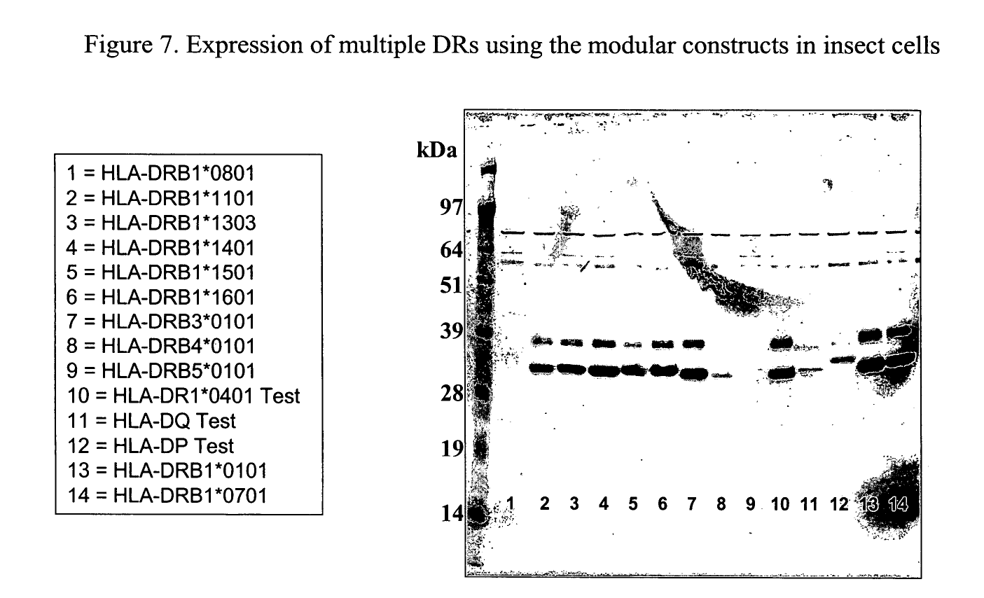

5 ug each of the plasmid DNAs for α and β subunit expression constructs were used for transfecting 2×106 HiFive cells plated in 150 mm tissue culture dishes. The plasmid DNAs were mixed with 50 ul of a liposome based transfection reagent, CellFectin (Invitrogen) in 2 ml of a serum-free medium, ESF-921 (ExpressionSystems LLC) and incubated for 20 min at RT for DNA-liposome complexes to form. The mix was added slowly to insect cells already plated in 150 mm dishes followed by a 4 hr incubation at RT with very gentle rocking (5 rpm). After incubation, 20 ml of fresh ESF-921 medium was added to each plate and allowed to incubate at 27 C for 3-5 days. The supernatant containing the secreted MHCs was harvested after 4 days and the cells fed with 25 ml of fresh medium. The second harvesting was done after 2 days following the re-feed (6 days post-transfection). The supernatants from the two harvests were analyzed for MHC expression by western blotting using anti-His and anti-Flag antibodies. The two supernatants were pooled before proceeding with purification. Using this transient transfection approach, MHC molecules of each class (DR, DP & DQ) using a test molecule of each class. The overall expression yield varies from 100-2000 ug/liter of the supernatant. See FIGS. 3 and 7.

### Example 3

### Expression in Mammalian Cells

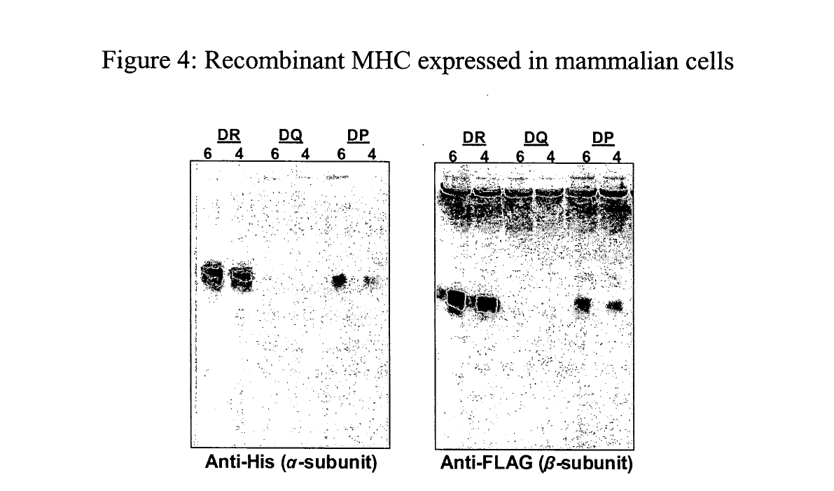

293 T cells (2×10⁵) were transfected with 20 ug each of the α and β subunit expression constructs using 100 ul of Lipofectamine. Medium was harvested after 4 days at 37C, 5% CO2, and replenished with fresh medium, again harvested after 2 days (total 6 days post transfection. The supernatants from the two harvests were analyzed for MHC expression by western blotting using anti-His and anti-Flag antibodies. Using this transient transfection approach, MHC molecules of each class (DR, DP & DQ) may be expressed using a test molecule of each class. The overall expression yield varies from 50-2000 ug/liter of the supernatant. See FIG. 4.

### Example 4

### Expression of Recombinant MHC Molecules of Each Class (HLA-DR, HLA-DP & HLA-DQ)

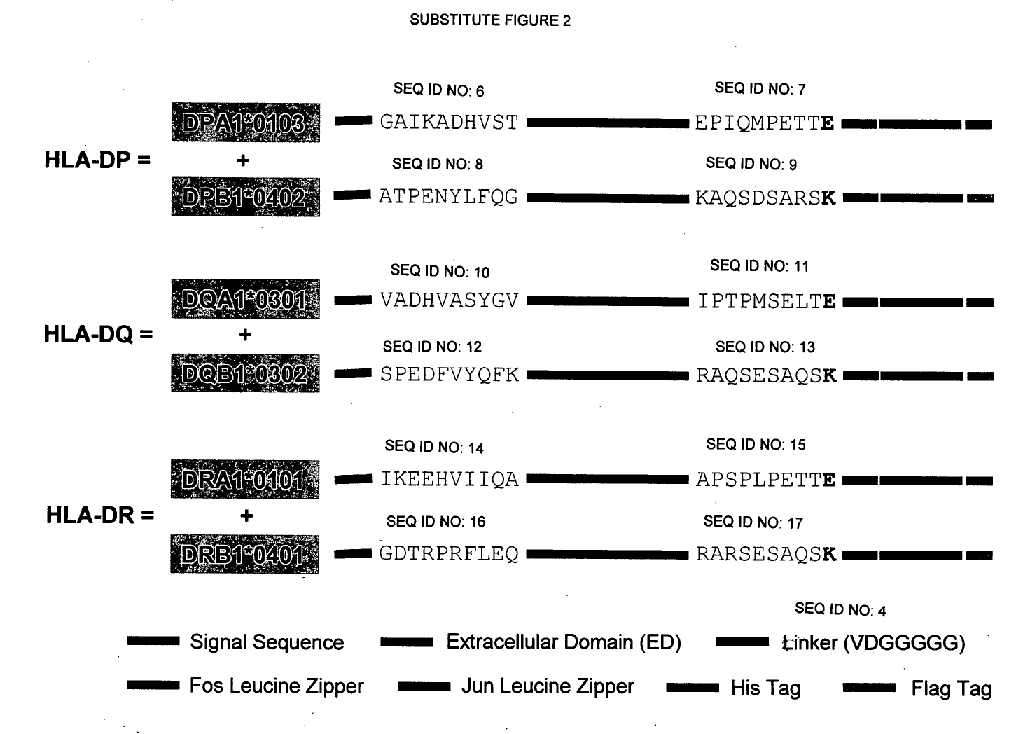

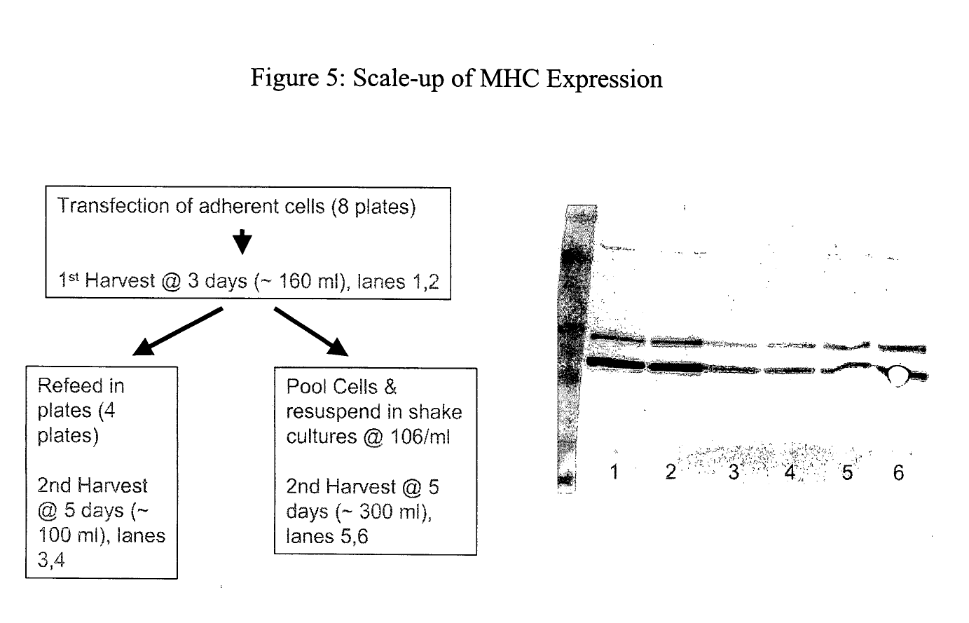

The recombinant MHC molecules were produced in insect cells using a non-lytic system involving transient transfection of cells with both α and β subunit expression constructs. The expression constructs for each of the subunits contained the respective extracellular domain attached at the c-terminus to the fos/jun leucine zipper dimerization domains and a his/flag tag sequence via a flexible linker (VDGGGGG) (SEQ ID NO: 4) as described in FIG. 2. The construct design for both α (A) and β (B) subunits of each of the test MHCs of DR, DP and DQ class. First and last 10 amino acids defining the boundaries of extracellular domain included in the constructs is presented. The α subunits contain a c-terminal fos LZ and a His tag, where as the β subunits contain c-terminal jun LZ and the flag tag. Co-transfection with plasmid DNAs encoding these two modular subunits yields heterodimeric MHC molecules in the medium supernatant driven by system appropriate signal sequence. The expression-reading frame was joined at the N-terminus with Honeybee Melittin signal sequence to facilitate efficient secretion of the correctly folded heterodimer MHCs in the media supernatant. The extracellular domains of the MHC molecules were amplified from corresponding cDNA clones obtained from ATCC and fused to synthetic DNAs containing the leucine zipper and the tag sequence using standard PCR based protocols. This modular insert was cloned downstream of the signal sequence in the expression vector. Scale-up of MHC expression was performed as shown in FIG. 5. Using this approach, the MHC proteins may be expressed in various expression systems as sampled in Table 1.

TABLE 1

| Expression | Possible Cell | | Vector | Signal | Stable or |
|---|---|---|---|---|---|
| System | Line(s) | Vector | Promoter | Sequence | Transient |
| Drosophila | S2 | PMT | Metallothionin- | BiP secretion | Transient; |
| | | (Invitrogen) | inducible | | stables |
| | | | | | possible |
| Pichia | GS115 Pichia | pPICZα | Methanol- | α factor | Stable |
| | pastoris | | inducible AOX | | |
| Mammalian | 293T | pSecTag2 | CMV | Igκ | Transient; |
| | | (Invitrogen) | | | stables |
| | | | | | possible |
| Insect | Hi5 | pMIB vector | pOPIE2 | Honeybee | Transient; |
| Select | | (Invitrogen) | constitutive | Melittin | stables |
| | | | | | possible |

### Example 5

### Purification of MHC Molecules

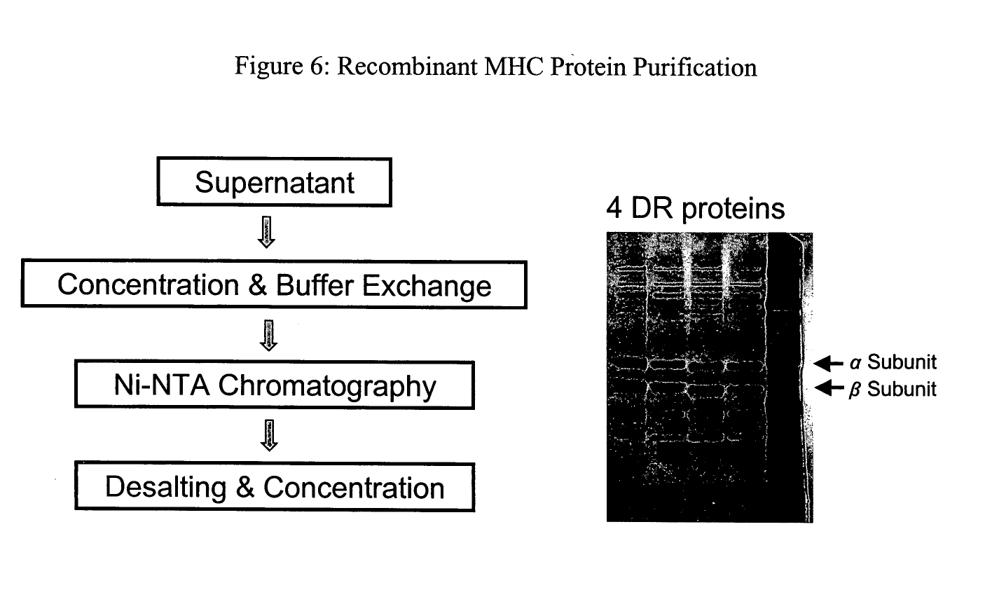

The recombinant MHC molecules expressed in cells may be purified using Ni-NTA affinity chromatography as described for the DR4 (DRA1*0101 & DRB1*0401) test molecule, FIG. 6. The supernatant was concentrated 20× using a tangential flow ultrafiltration cassette (Millipore, Pelicon) with a MWCO of 10000 d. The concentrated supernatant was buffer exchanged with binding buffer (50 mM Tris-HCl, pH 8.0 & 500 mM NaCl) using the same ultrafiltration device. Ni-NTA agarose beads were mixed with this in the presence of 20 mM imidazole and 10% glycerol. After 4-6 hrs incubation at 4C with constant mixing, the slurry was poured into a chromatography column and washed with 50 mM Tris-HCl, pH 8.0, 500 mM NaCl and 30 mM imidazole followed by one more wash with 50 mM Tris-HCl, pH 8.0 & 1.0 M NaCl. The bound protein was eluted with 50 mM Tris-HCl, pH 8.0, 100 mM NaCl and 250 mM imidazole in 10% glycerol. The eluted protein was buffer exchanged into PBS with 10% glycerol. This yields a partially purified (˜50%) and concentrated preparation is stable and may be used in directly in all binding assays and surface captures.

### Example 6

### MHC-Peptide Binding Assays

The recombinant MHC molecules produced as described above may be used to test the binding of various peptides either in solution or as captured or arrayed on a solid surface. The peptide binding may be determined in a direct binding experiment where the peptide is labeled (e.g., with radioactivity, fluorescence, biotin etc.) or in a competition format where the reference peptide is labeled but the test peptide(s) are unlabeled.

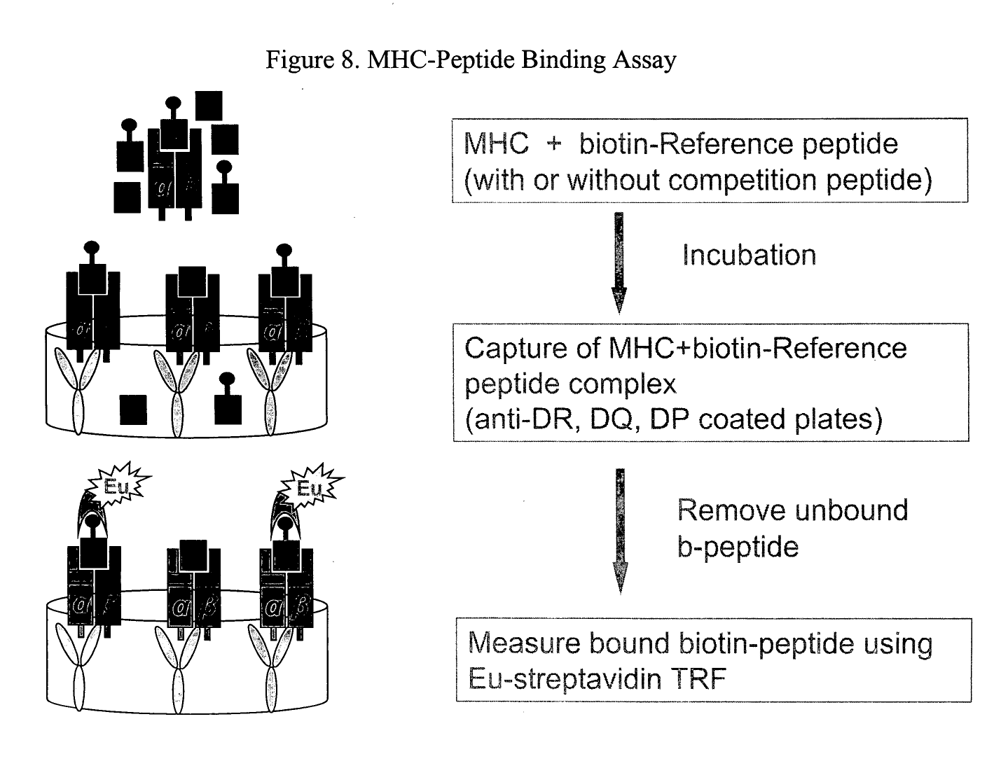

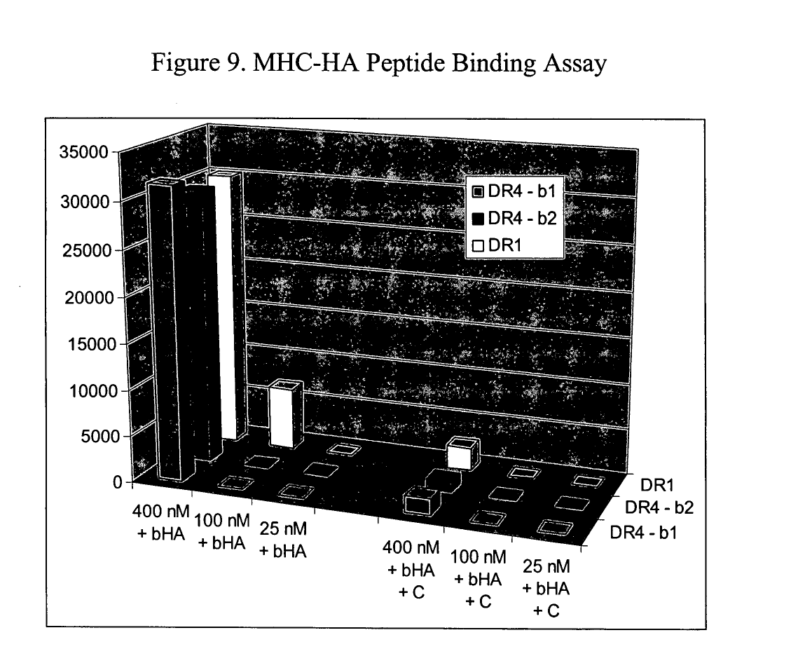

For detection of peptide binding the MHC and peptide were mixed together in PBS, pH 7.2 containing 1 mM PMSF and 1 mM EDTA. After overnight incubation at 37C, the MHC-peptide complex was captured on plates coated with an anti-MHC antibody. The unbound peptide was removed by several washes and the amount of MHC bound biotin label was detected using Eu-sterptavidin time-resolved fluorescence (Delfia® assay), FIG. 8. As shown in FIG. 9, the biotin labeled HA peptide (biotin-Ahx-Ahx-PKYVKQNTLKLAT (SEQ ID NO: 5) with Ahx=aminohexanoic acid spacer) binds specifically and competitively with both DR4 and DR1. The binding with b-HA may be effectively competed out with 10-fold excess of unlabelled peptide.

Peptide binding affinities may be determined by incubation of the labeled (biotin, fluorescence, radiolabeled) peptide over the surface and then evaluating the amount of bound peptide using appropriate detection methodologies. A dose response experiment using multiple concentrations of the peptide in sequence may be done to evaluate the specific ED50 values for each peptide-MHC combination. The dissociation rates may also be calculated by monitoring the label after loading as a function of time. Fluorescently labeled (for example, FAM, fluorescein, Alexa, Cy dyes) peptides would be the preferred format for this analysis. In another embodiment, a competition based method may also be used to analyze the relative binding affinities of unlabelled peptides using a single labeled reference peptide pre-loaded onto MHC molecules as long as the reference peptide binds to all of them. An SPR based approach may also be used to study the binding interactions using an SPOT-Matrix method.

### Example 7

### Production of MHC Protein Arrays

MHC protein arrays having about 2 to about 1000 different MHCs may be prepared by surface capture on glass, plastic, nitrocellulose, hydrogels or other derivatized surfaces using either direct binding or binding via specific interactions. Specific interactions include but are not limited to antibodies against a common tag, streptavidin for a biotinylated MHC or protein A or albumin or Fc for a fusion MHC). Control proteins and multiples of same MHCs may be used as internal controls.

In some cases, every pad on the array has the same capture molecule, and each MHC construct has the same capture sequence. In this embodiment, the array is used more as a general affinity capture surface, in a manner similar to phage display panning. In this embodiment, the MHC constructs are bound to the array (which can also be a continuous surface, rather than spatially separate addresses) and test molecules added. Washing and competitive assays may be done to test for protein-protein interactions and affinity.

### Example 8

### Method of Studying MHC-Peptide Binding Interaction

The present invention may be used to study MHC-peptide binding interaction in an array format where either MHC or the peptide would be used as the arrayed partner.

The MHC bioarray may comprise more than one MHC molecule, more preferably 2-10000, and even more preferably from 2-100. Either the MHC proteins or the peptides may be attached to a solid surface in an ordered format. The attached molecules may be selected based on: 1) a specific population prevalence; 2) a specific disease state association/disease susceptibility; 3) a specific structural subclass(s) of MHCs; or 4) other criteria. The MHCs may be natural MHC isolated from cells, or recombinant produced with a natural ectodomain sequence or recombinant with a modified ectodomain sequence.

The peptide bioarrays may comprise of a selection of 2-100000, more preferably a range of 2-1000. In a preferred embodiment, the peptides are attached to a solid surface in an ordered format. The peptides may be selected, for example, from the following groups: 1) a peptide scan of one or more protein sequences; 2) randomly selected from genome sequencing; 3) peptides containing sequence similar to those occurring in natural proteins with one or more modifications; 4) completely synthetically created sequences; 5) other criteria. The peptides may be 6-30 amino acids long, with preferred range being 8-16. The peptides may have spacer amino acids, may be attached to surface via biotin or directly coupled to surface during synthesis or attached using other chemistries.

For MHC bioarrays, the bioarray may be reacted with labeled/unlabeled specific peptides or population of peptides to characterize the binding interaction. For peptide bioarrays, the bioarray may be reacted with labeled/unlabeled specific MHCs or population of MHCs to characterize the binding interaction.

The interactions (from both formats) may be used to identify immunogenic epitopes on the proteins, de-immunize protein sequences, select populations with specific MHCs that could be used for clinical trials or therapeutic use, improve the potency of a vaccine, etc.

### Example 9

### Predicting Immunogenicity of a Therapeutic Candidate

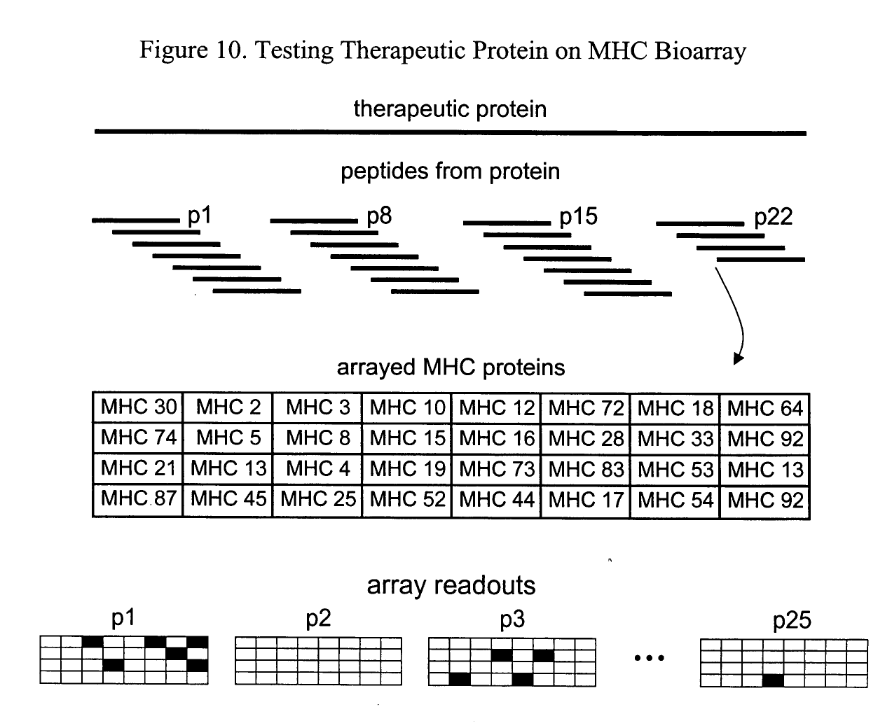

As shown in FIG. 10, a therapeutic protein may be analyzed using a MHC bioarray of the present invention. The target molecule may be expressed as multiple overlapping peptides, preferably from 8 to 16 amino acids in length. These peptides may be run over a MHC bioarray as described herein to study the MHC-peptide binding.

The MHC bioarray may be optimized to a target population for the therapeutic, such as people with type II diabetes in the US population; Alzheimer's patients; MS patients, arthritic patients; etc.

### Example 10

### Bioarray of Peptides

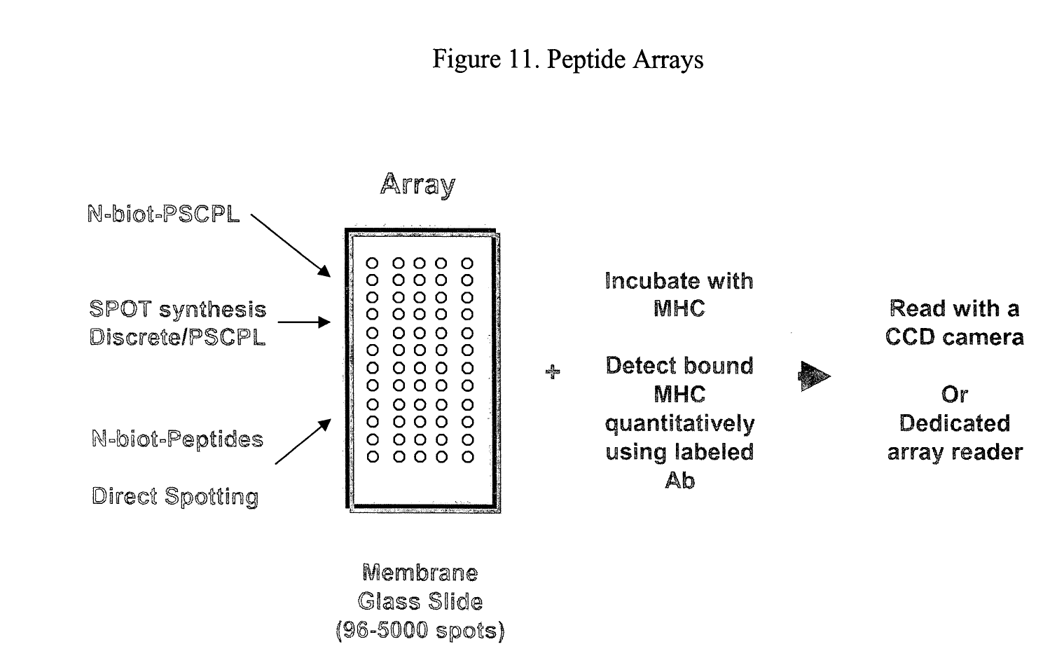

As shown in FIG. 11, an the present invention may be used with an array of peptides. The peptides may be bound to the surface in an oriented manner and the binding of individual MCHs may be evaluated. In a preferred embodiment, binding is evaluated using either an SPR based method or detection using an MHC specific labeled antibody.

In a preferred embodiment, a peptide array may be use for the analysis of a smaller number of MHC molecules against a larger set of peptides. In a preferred embodiment, libraries of different candidate proteins may be used. However, as will be appreciated by those in the art, different members of the library may be reproduced or duplicated, resulting in some libraries members being identical.

### Example 11

### Predicting Immune Response to Vaccine Candidate

The present invention may be used to predict the immune response to a vaccine candidate. As shown in FIG. 10, the proteins of the vaccine candidate may be expressed as multiple overlapping peptides, preferably from 8 to 16 amino acids in length. The peptides may then be run over a MHC bioarray. Preferably, the bioarray will include a combination of MHC constructs to represent over 99% of the target population. Analysis of the binding may then be used to predict the effectiveness of the candidate vaccine.

Whereas particular embodiments of the invention have been described above for purposes of illustration, it will be appreciated by those skilled in the art that numerous variations of the details may be made without departing from the invention as described in the appended claims. All references cited herein are incorporated entirely by reference.

We claim:

1. A method of screening for binding of candidate peptides to an MHC construct comprising:
a) contacting a first component comprising at least one candidate peptides with a second component comprising at least one MHC construct, wherein one of said components is immobilized on a solid support; and
b) determining the presence or absence of binding of said peptide and said MHC construct.

2. A method according to claim 1 wherein said peptides are immobilized on said support.

3. A method according to claim 2, wherein the second component comprises a library of MHC constructs.

4. A method according to claim 1 wherein said MHC construct is immobilized on said support.

5. A method of claim 4, wherein the first component comprises a plurality of candidate peptides.

6. A method according to claim 2 or 4 wherein said support comprises microspheres.

7. A method according to claim 2 wherein said MHC construct is labeled.

8. A method according to claim 4 or wherein said peptides are labeled.

9. A method according to claim 7 or 8 wherein said labels are fluorophores.

10. A method according to claim 7 or 8 wherein said labels are secondary labels.

11. A method according to claim 10 wherein said secondary labels are epitope tags.

12. A method according to claim 1 wherein a plurality of MHC constructs are immobilized on said support.

13. A method according to claim 1 wherein said MHC construct comprises an attachment linker and an MHC protein.

14. A method according to claim 1 wherein said MHC construct comprises a label and an MHC protein.

15. A method according to claim 14 wherein said label is a fluorescent protein.

16. A method according to claim 1 further comprising identifying the sequence of a peptide bound to said MHC construct.

17. A method according to claim 16 further comprising adding said peptide to a cell comprising an MHC protein and assaying for activity.


SEQUENCE LISTING (17 sequences)

SEQ ID NO: 1 - protein, 11 aa, Artificial
  Synthetic
MGXXXXNGGPP

SEQ ID NO: 2 - protein, 5 aa, Artificial
  Synthetic
GSGGS

SEQ ID NO: 3 - protein, 4 aa, Artificial
  Synthetic
GGGS

SEQ ID NO: 4 - protein, 7 aa, Artificial
  Synthetic
VDGGGGG

SEQ ID NO: 5 - protein, 13 aa, Artificial
  Synthetic
PKYVKQNTLKLAT

SEQ ID NO: 6 - protein, 10 aa, Artificial
  Synthetic
GAIKADHVST

SEQ ID NO: 7 - protein, 10 aa, Artificial
  Synthetic
EPIQMPETTE

SEQ ID NO: 8 - protein, 10 aa, Artificial
  Synthetic
ATPENYLFQG

SEQ ID NO: 9 - protein, 10 aa, Artificial
  Synthetic
KAQSDSARSK

SEQ ID NO: 10 - protein, 10 aa, Artificial
  Synthetic
VADHVASYGV

SEQ ID NO: 11 - protein, 10 aa, Artificial
  Synthetic
IPTPMSELTE

SEQ ID NO: 12 - protein, 10 aa, Artificial
  Synthetic
SPEDFVYQFK

SEQ ID NO: 13 - protein, 10 aa, Artificial
  Synthetic
RAQSESAQSK

SEQ ID NO: 14 - protein, 10 aa, Artificial
  Synthetic
IKEEHVIIQA

SEQ ID NO: 15 - protein, 10 aa, Artificial
  Synthetic
APSPLPETTE

SEQ ID NO: 16 - protein, 10 aa, Artificial
  Synthetic
GDTRPRFLEQ

SEQ ID NO: 17 - protein, 10 aa, Artificial
  Synthetic
RARSESAQSK